(19) United States
(12) Patent Application Publication
(10) Pub. No.: US 2008/0247736 A1
(43) Pub. Date: Oct. 9, 2008

(54) Device for Emanating an Active Agent

(75) Inventors: Paul Marrs, Hull (GB); Malcolm Tom McKechnie, Hull (GB)

Correspondence Address: NORRIS, MCLAUGHLIN & MARCUS, 875 THIRD AVE, 18TH FLOOR, NEW YORK, NY, 10022, US

(73) Assignee: RECKITT BENCKISER (UK) LIMITED, Slough, Berkshire (GB)

(21) Appl. No.: 12/088,148

(22) PCT Filed: Sep. 28, 2005
(86) PCT No.: PCT/GB06/03605

§ 371 Date: May 23, 2008

(30) Foreign Application Priority Data
Sep. 28, 2005 (GB) 0519717.3

(51) Int. Cl. A61L 9/03; F24J 1/00

(52) U.S. Cl. 392/387; 239/37; 126/263.01; 392/394

(57) ABSTRACT

A device for emanating an active agent to the atmosphere comprising a first container holding a first liquid, a second container holding a second liquid, a collector, and an active agent, wherein an outlet from each container deposits the first liquid and the second liquid onto the collector whereby the liquids mix and react to generate heat and thereby volatilise the active agent

The present invention relates to a device that enables the emanation of a volatile active agent, such as a deodorising, sanitising, air freshening, aromatherapy, therapeutic, pesticidal or insect repellent composition, into the surrounding atmosphere without requiring separate heating and/or electrical means.

The use of various devices for the diffusion of volatile active agents, for example air-freshening, deodorising, pesticidal or insect repellent products, into the atmosphere has become increasingly popular in recent years.

For example, air-freshening devices or deodorisers are currently used in many households to mask bad odours, or to impart fragrances to the ambient air. Various types of devices are known for the diffusion of volatile active agents into the surroundings.

Devices for providing and dispensing volatile active agents are well-known in the art. Such devices can include room fresheners or fragrancing articles, vaporizers for humidification or dispensing and dispersal of therapeutic vapours, incense sticks, fragrancing or insect-repelling candles. Such devices generally involve utilization of some source of heat which promotes the volatilization of the active agents to be dispensed and, of course, some source of the volatile or volatilizable materials themselves.

Some fragrancing devices, such as candles and incense sticks, involve use of an open flame or an active combustion reaction to provide the source of heat which promotes volatilization. Besides the obvious drawbacks of the hazards of using such types of heat sources, arrangements which involve flames or combustion are not especially portable and cannot be used, for example, in automobiles or around flammable materials.

Other types of dispensing devices for volatiles use electrical energy as a heat source, for example plug-in room air-fresheners. The amount of electrical energy required to operate devices of this type renders such devices relatively non-portable.

Given this situation, there is a continuing need to provide dispensing devices in forms which are safe, portable, readily consumer activable and controllable so as to be especially effective at dispensing the desired volatile materials in suitable amounts, at the desired time and over an acceptably prolonged period of time.

Accordingly, the present invention provides a device for emanating an active agent to the atmosphere comprising a first container holding a first liquid, a second container holding a second liquid, a collector, and an active agent, wherein an outlet from each container deposits the first liquid and the second liquid onto the collector whereby the liquids mix and react to generate heat and thereby volatilise the active agent.

An example of the device in accordance with the present invention will now be described with reference to the accompanying drawings, in which:

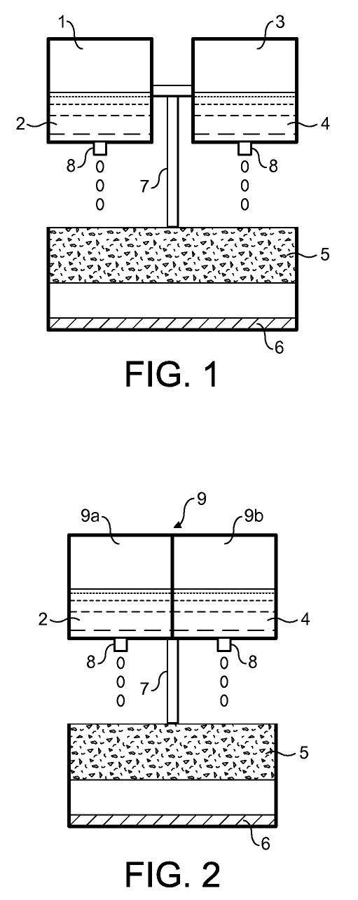

FIG. 1 is a perspective view of one preferred embodiment of the present invention

FIG. 2 is a perspective view of a second preferred embodiment of the present invention.

FIG. 1 shows containers 1 and 3 with a first liquid 2 in container 1 and a second liquid 4 in container 2.

By “container”, we simply mean a region where the particular liquid is kept until use.

The containers can be two separate containers 1,3 as shown in FIG. 1 or can be one container 9 with two separate compartments 9a,9b as shown in FIG. 2, one for the first liquid and the other for the second liquid.

The containers are generally made of a plastic material. In a preferred embodiment the material is polyethylene or polypropylene. The containers are designed to be durable. The containers can be made of any suitable material.

In one embodiment the liquids can be sealed in the containers by a sealing means. In a preferred embodiment the containers are sealed in such a way to allow air to circulate within the containers. Thus, preferably the sealing means is a membrane, more preferably a gortex-type membrane. A person skilled in the art will understand that any suitable sealing means can be used.

The containers may, however, also have an air-tight sealing means, such as a lid. In a preferred embodiment when an air-tight sealing means is used as gas generating means will be incorporated within the container.

The container may also have a sealing means which can be easily opened and closed by the user to enable them to add additional liquids to the containers if and when required.

The containers are preferably at least partially transparent to allow the liquids of the present invention to be viewed from the package exterior. For example, the package may be formed of a transparent material or alternatively or additionally, the container may be provided with a transparent window through which the liquids are visible. Such features will enable the user to see when the liquids in the container need replenishing and/or the device replaced.

The containers 1,3 are attached to a supporting means 7. The containers 1, 3 can be permanently attached to the supporting means or relesably attached to the supporting means 7. The supporting means can be made of any suitable material. Preferably the supporting means are generally made of a plastic material. In a preferred embodiment the material is polyethylene or polypropylene. More preferably the supporting means 7 is made of the same material as the containers 1,3.

As outlined above the device of the present invention may be reused. Thus after the first and/or second liquids in the containers has been used up, the containers may be replenished with additional liquids. The replenishment of the containers may, if desired, be carried out by the user. The containers can be removed from the supporting means to assist the user in replenishing the containers.

The supporting means 7 is attached to a collector 5.

In a preferred embodiment the collector is a porous plate.

In use, the porous substrate allows the impregnated composition to be released into the surrounding atmosphere in vapour form.

The porous substrate preferably has a maximum pore size of up to 100 microns, preferably up to 90 microns, and more preferably, up to 80 microns. The mean pore size may be 5 to 80 microns, preferably 10 to 50 microns, and more preferably, 15 to 30 microns. Pore size may be measured using microscope techniques.

The substrate may have a porosity or liquid loading capacity of from 60 to 80%, preferably 65 to 75%.

The substrate may be formed of any suitable material. For example, the substrate may be formed of plaster, silica and/or ceramic material. Preferably, the substrate comprises a synthetic polymer, such as a polyolefin, Suitable polyolefins include polypropylene, high density polyethylene (HDPE), linear low density polyethylene (LLDPE) or low density polyethylene (LDPE). Suitable substrates include Accurel MP (trademark) from Membrana GmbH.

Once impregnated the liquid composition may fill the loading capacity of the substrate by at least 10%, preferably, at least 20%, more preferably, at least 30%, yet more preferably, at least 40%, and even more preferably, at least 50%. Thus, the liquid composition may form 30 to 60 wt % of the overall weight of the impregnated surface, and more preferably 35 to 45 weight. With use, at least a portion of the impregnated liquid composition will evaporate from the surface.

To prevent any of the mixed liquid composition which does not evaporate into the atmosphere from spilling out of the collector a receiving plate 6 can be located underneath the collector to receive any excess liquid. The receiving plate can be attached to the collector. This plate 6 can then be emptied to remove any excess liquid by the user or can be left as the excess liquid will simply evaporate into the atmosphere over a period of time.

The receiving plate 6 is generally made of a plastic material. In a preferred embodiment the material is polyethylene or polypropylene. The receiving plate 6 is designed to be durable. The receiving plate 6 can be made of any suitable material.

The containers or each compartment in the container if only one container is used has an outlet 8 to enable the first and second liquids to be delivered to the collector 5.

Each container or each compartment in the container may have an outlet 8 or where one outlet can be present which can dispense both the first and the second liquids.

In a preferred embodiment the containers are provided with actuation means for delivering a predetermined volume of each of the first liquid and second liquid to the collector. The actuation means can be a moveable piston which can be manually operated or the containers themselves can be flexible such that they can be squeezed by a user to deliver a predetermined volume.

In a preferred embodiment the first liquid comprises a reducing agent. Suitable reducing agents include sulfides, sulfites, sulfates, oxazolidines, bisulfates, ascorbic acid, oxalic acid, iodides, ferrous ammonium sulphate, and thiosulfates, preferably alkali metal thiosulfates. The alkali metal can be for example Li, Na or K. Examples include sodium thiosulfate, sodium bisulfate, sodium sulfite, potassium iodide. Most preferred are thiosulfates, and most preferred is sodium thiosulfates. The reducing agents should be chosen to ensure that the oxidising agent behaves thus.

The preferred amount of reducing agent in the first liquid is preferably such that there is sufficient reducing agent present to reduce all, substantially all, or at least most, of the oxidising agent present in the second liquid, whilst providing the sufficient generation of heat to volatilise the active agent. Most preferably the concentration of reducing agent is the same, or substantially the same, as the amount of oxidising agent present in the second liquid. Even more preferably, the reducing agent is present in the first liquid at a concentration of from 2 to 20 weight % (where weight percent of reducing agent is the concentration of reducing agent in the first liquid, i.e. the weight percent of the reducing agent in the first liquid), preferably from 4 to 15 weight %, more preferably from 5 to 10 weight % and even more preferably in an amount of about 5 weight %

In a preferred embodiment the second liquid comprises an oxidising agent. Suitable oxidising agents include both peroxygen-based oxidising agents and hypohalite-based oxidising agents. Examples include hydrogen peroxide, hypochlorous acid, hypochlorites, hypocodites, and percarbonates. Also included are alkali metal chlorites, hypochlorites, for example sodium chlorite and sodium hypochlorite. Hydrogen peroxide precursors such as peroxygen bleaching agents can also be used, for example alkali metal perborates and percarbonates, for example, sodium percarbonate and sodium perborate. However, particularly preferred as the oxidising agent are peroxides, most particularly hydrogen peroxide. Thus, in a particularly preferred embodiment of the invention, the second liquid comprises hydrogen peroxide.

Hydrogen peroxide is a chemical that has particular user compliance considerations. It is a relatively strong oxidising agent and as such, concentrated hydrogen peroxide solutions should not be in direct contact with the user. Although in the present invention, oxidising agent combines with the reducing agent to generate heat, the presence of relatively high concentration oxidising agents even as part of one of the liquids could prove dangerous. For instance, if the oxidising and reducing agents are not completely mixed, there exists the possibility of non-reduced oxidising agents being touched by the user. Moreover, should the mixing or combining mechanism of the two components malfunction in any way, it is again possible for non-reduced oxidising agent to be present. Hence, it is preferable for the concentration of oxidising agent to be as low as possible, whilst still retaining the ability to react with the reducing agent and thus generate heat.

Preferably, therefore, the oxidising agent is present in the second liquid at a concentration of from 2 to 20 weight % (where weight percent of oxidising agent is the concentration of oxidising agent in the second liquid, i.e. the weight percent of the oxidising agent in the second liquid), preferably 4 to 15 weight %, more preferably from 5 to 10 weight % and even more preferably in an amount of about 7 weight %.

In a preferred embodiment when a sufficient amount of reducing agent, for example sodium thiosulfate, and oxidising agent, for example, hydrogen peroxide, are mixed together, a Redox reaction occurs. Thus the hydrogen peroxide brings about the oxidation of sodium thiosulfate at the same time as the sodium thiosulfate brings about the reduction of the hydrogen peroxide. This Redox reaction results in the generation of heat. Sufficient heat is produced to enable the active agent to volatilise.

A person skilled in the art will understand that the levels of reducing and oxidising agents can be tailored to deliver quicker heat, longer lasting heat, more heat, less heat and variations along that theme by altering the amount of reducing and/or oxidising agent present in the liquids.

Suitably, in use, appropriate relative amounts of reducing and oxidising agents are mixed such that they mix in the amounts required to generate heat and thereby volatise the active agent. In preferred embodiments, the compounds are preferably mixed in a ratio between 10:1 and 1:10 by weight, more preferably between 5:1 and 1:5 by weight, most preferably between 2:1 and 1:2 by weight, for example, approximately equal amounts.

A person skilled in the art will understand that any mixture of two compounds that can produce an exothermic reaction, i.e. produce heat, can be used in the present invention.

The active agent can be present on the collector and/or in the first liquid and/or in the second liquid. In a preferred embodiment the first liquid and/or the second liquid comprises the active agent. More preferably the first liquid comprises the active agent.

The active agent or desired base can be an air-freshening, deodorising, pesticidal and/or insect repellent base.

When an air freshening composition is employed, the active agent includes an air-freshening or perfume base. Any perfume base that is currently used in perfumery may be employed. Thus, the perfume base may be formed of discreet chemicals. More often, however, the base will be a mixture of volatile liquid ingredients of natural or synthetic origin. The nature of these ingredients may be determined with reference to specialised books of perfumery, such as “Perfume and flavour Chemicals” (S. Arctander, Montclair N.J., USA 1969), “Perfumery” (Wiley-Intersciences, New York, USA 1994) or similar references.

A perfume base may also be included in any deodorising, aromatherapy, therapeutic, pesticidal or insect repellent composition employed. For example, insect repellent fragrant materials may be used, such as citronella oil, thus providing a device and method for repelling insects.

The active agent is present in an amount of from 2 to 20 weight %, preferably from 4 to 15 weight %, more preferably from 5 to 10 weight % and even more preferably in an amount of about 5 weight %

A catalyst that increases the rate of the redox reaction between the two liquids can also be present. The catalyst can be present on the collector and/or in the first liquid and/or in the second liquid. In a preferred embodiment the catalyst is present in the first liquid and/or on the collector. More preferably the collector comprises the catalyst for the reaction between the liquids. Preferably, the catalyst comprises a metal-containing ion, more preferably a transition metal-containing ion, for example containing an ion of manganese, copper, molybdenum, or tungsten, together with an alkali or alkaline earth metal, such as sodium. More preferably, the catalyst is a tungstate compound, although other ions comprising a transition metal ion and oxygen, e.g. manganese, copper, or molybdenum with oxygen, can be used. Yet more preferably, the catalyst is an alkali metal tungstate (e.g. contains the WO₄²⁻ ion), most preferably sodium tungstate (e.g. Na₂WO₄), suitably in the form sodium tungstate dehydrate (e.g. Na₂WO₄.2H₂O).

When the oxidising agent is a hydrogen peroxide precursor such as peroxygen bleaching agents, for example an alkali metal perborate or percarbonate a bleach activator such as tetraacetyl ethylene diamine (TAED) or nonanoyloxybenzene sulfonate (NOBS)and mixtures thereof is preferably present. The bleach activator will assist in accelerating the break down of the bleach activator to the corresponding peroxy acid, for example, alkali metal perborate or percarbonate to hydrogen peroxide.

Preferably, the catalyst is present in an amount sufficient to catalyse the reaction between the two liquids. More preferably, the catalyst is present to a maximum of 2 weight %, even more preferably to a maximum of 1 weight %, yet more preferably in the range 0.01 to 0.5 weight %, even more preferably in the range 0.1 to 0.5 weight %, for example at about 0.2 weight % or at about 0.5 weight %.

The first liquid may also further comprise a basic buffering system, such as amino-alcohol compounds, for example 2-aminoethanol (MEA), in an amount of from 1 to 10%, preferably from 2 to 5 weight %, most preferably about 3.5 weight %.

The first liquid may also further comprise an oil solubiliser such as DPnB or hexylene glycol, in an amount of from 1 to 10%, preferably from 2 to 5 weight %, most preferably about 3 weight %.

### EXAMPLE

The containers 1, 3 in FIG. 1 and the compartments 9a, 9b in FIG. 2 can contain the following:

The first liquid comprising:
5 weight % Sodium thiosulphate
0.5 weight % Sodium Tungstate
3.5 weight % MEA
3 weight % DPnB
5 weight % Fragrance
83 weight % Deionised water

was added to a first container.

The second liquid comprising:
7 weight % hydrogen peroxide
93 weight % deionised water

was added to the second container.

A predetermined volume of each of the first and second liquid is then delivered to the porous collector plate via an actuation means.

A Redox reaction then occurs which generates sufficient heat to enable the fragrance present in the first liquid to evaporate into the atmosphere.

1. A device for emanating an active agent to the atmosphere comprising a first container holding a first liquid, a second container holding a second liquid, a collector, and an active agent, wherein an outlet from each container deposits the first liquid and the second liquid onto the collector whereby the liquids mix and react to generate heat and thereby volatilise the active agent.

2. A device according to claim 1 wherein the first liquid and/or the second liquid comprises the active agent.

3. A device according to claim 1 wherein the collector is in thermal contact with the active agent.

4. A device according to claim 1, wherein the collector is a porous plate.

5. A device according to claim 1, wherein the first liquid comprises a reducing agent and the second liquid comprises an oxidising agent.

6. A device according to claim 5 wherein the first liquid comprises an alkali metal thiosulfate or bisulfate.

7. A device according to claim 5 wherein the second liquid comprises aqueous hydrogen peroxide.

8. A device according to claim 1, wherein the first liquid and/or second liquid comprises a catalyst for the reaction between said liquids.

9. A device according to claim 1, wherein the collector comprises a catalyst for the reaction between said liquids.

10. A device according to claim 1, which device comprises an actuation means for delivering a predetermined volume of each of the first liquid and second liquid to the collector.

11. A device according to claim 1, wherein the active agent is a deodorising, sanitising, air freshening, aromatherapy, therapeutic, pesticidal or insect repellent composition.
